(19) United States
(12) Patent Application Publication
(10) Pub. No.: US 2025/0025038 A1
(43) Pub. Date: Jan. 23, 2025

(54) VIDEO LARYNGOSCOPE WITH AUTOMATIC BLADE DETECTION

(71) Applicant: Covidien LP, Mansfield, MA (US)

(72) Inventors: Alexandra R. Hause, Denver, CO (US); Derek S. Tata, Longmont, CO (US); Xin-An Chen, Broomfield, CO (US)

(21) Appl. No.: 18/765,969

(22) Filed: Jul. 8, 2024

Related U.S. Application Data
(60) Provisional application No. 63/514,241, filed on Jul. 18, 2023.

(51) Int. Cl. A61B 1/267; A61B 1/00; A61B 1/05

(52) U.S. Cl. CPC A61B 1/267; A61B 1/000096; A61B 1/00052; A61B 1/05

(57) ABSTRACT

A video laryngoscope with blade identification is disclosed. The video laryngoscope may be capable of identifying a blade that has been coupled to the video laryngoscope. Identification of the blade may be based on an image from a camera of the video laryngoscope. The blade identification may be automatic. Blade identification may be performed via image recognition rules and/or machine learning (ML) models. Additionally, an image may be the only information used to determine blade identification. Based on the blade identification, various settings of the video laryngoscope may be adjusted (e.g., lighting or camera settings). Determined blade identification may be sent to a facility computer for maintaining patient records, procedure records, and/or inventory. Additionally, blade identification may be used in determining a video classification of intubation (VCI) score.

### CROSS-REFERENCE TO RELATED APPLICATION

The present application claims the benefit of U.S. Provisional Patent Application Ser. No. 63/514,241, filed on Jul. 18, 2023, the entire content of which is incorporated herein by reference.

### INTRODUCTION

Laryngoscopes are commonly used during intubation of a patient (e.g., an insertion of an endotracheal tube into a trachea of the patient). In video laryngoscopy, a medical professional (e.g., a doctor, therapist, nurse, clinician, or other practitioner) views a real-time video feed, captured via a camera of the video laryngoscope, of the patient's larynx on a display screen to facilitate navigation and insertion of tracheal tubes within the airway.

Laryngoscopes may be fitted with different blades. Blades vary in shapes and sizes to accommodate for different patient anatomy. For example, a pediatric patient may use a smaller blade to fit a smaller airway, and an adult patient may use a larger blade to fit a larger airway. Blades with different curvature may provide ease of operability and/or different viewing angles of patient anatomy when placed inside of the airway.

It is with respect to this general technical environment that aspects of the present technology disclosed herein have been contemplated. Furthermore, although a general environment is discussed, it should be understood that the examples described herein should not be limited to the general environment identified herein.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

Among other things, aspects of the present disclosure include systems and methods for blade detection via a video laryngoscope. In an aspect, a method for blade size detection by a video laryngoscope is disclosed. The method includes acquiring an image, by a camera of the video laryngoscope, the image including a portion of a blade coupled to the video laryngoscope. Based on the acquired image, the method includes automatically identifying the blade. The method further includes, in response to the identification of the blade, adjusting at least one setting of the video laryngoscope.

In an example, the at least one setting of the video laryngoscope is a camera setting, and wherein the camera setting is one of: gain; color; backlight brightness; or crop region. In another example, the at least one setting of the video laryngoscope is a brightness of lighting provided by the video laryngoscope. In a further example, the blade identification includes at least one of a size of the blade or a curvature of the blade. In yet another example, automatically identifying the blade includes providing the image as an input to a trained machine-learning (ML) model. In still a further example, the acquired image is an uncropped image of the camera, and wherein a cropped version of the acquired image is displayed on a display of the video laryngoscope.

In another aspect, a video laryngoscope is disclosed. The video laryngoscope includes a handle portion; a display screen coupled to the handle portion; a blade portion, coupled to the handle portion and coupled to a blade, configured to be inserted into a mouth of a patient; a camera, positioned at a distal end of the blade portion, that acquires a video feed while the video laryngoscope is powered on; a memory storing a trained machine-learning (ML) model; and a processor. The processor operates to receive an image of the video feed from the camera, the image including a portion of the blade. The processor further operates to provide the image as input to the trained ML model. Additionally, the processor operates to receive an output from the trained ML model in response to the input image, wherein the output includes an identification of the blade. The processor also operates to transmit the blade identification to a remote device.

In an example, the remote device is a hospital computer for maintaining at least one of inventory or patient records. In another example, the video laryngoscope further includes lighting on the blade portion, and wherein the processor further operates to adjust at least one of a lighting setting of the lighting or a camera setting of the camera, based on the blade identification. In a further example, the blade identification is displayed on the display screen. In yet another example, the blade identification is determined automatically. In still a further example, the determination of the blade identification is based only on the image.

In another aspect, a method for blade size detection by a video laryngoscope is disclosed. The method includes acquiring one or more images using a camera of the video laryngoscope. Based on the one or more images, the method includes identifying a blade coupled to the video laryngoscope; determining a percentage of glottic opening (POGO) visible; and determining an outcome of intubation. Based on the blade identification, the POGO visible, and the outcome of intubation, the method further includes determining a video classification of intubation (VCI) score.

In an example, the blade identification is based on a first image of the one or more images that includes a portion of a blade coupled to the video laryngoscope; wherein the POGO visible is based on a second image of the one or more images that includes the portion of the blade and patient anatomy; and wherein the outcome of intubation is based on a third image. In another example, the one or more acquired images are provided as inputs into a trained machine learning (ML) model, and wherein at least one of the blade identification or the POGO visible are received as outputs of the trained ML model.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a schematic of an example patient environment including a video laryngoscope.

FIG. 2 is a perspective view of the video laryngoscope of FIG. 1.

FIGS. 3A-3E show example blades for the video laryngoscope of FIG. 1.

FIG. 4 is a block diagram of components of the video laryngoscope.

FIGS. 5A-5I show example display screens of a video laryngoscope with blade size detection.

FIGS. 6A-6B show example methods for blade detection with a video laryngoscope.

FIG. 7 shows another example method for blade detection with a video laryngoscope.

FIG. 8 shows an example method for determining a video classification of intubation score with a video laryngoscope.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

Video laryngoscopes are commonly used during intubation of a patient (e.g., an insertion of an endotracheal tube into a trachea of the patient). During intubation, the patient's airway and larynx may be visualized by a medical professional (e.g., a doctor, therapist, nurse, clinician, or other practitioner), such as via video laryngoscopy. In video laryngoscopy, the medical professional may view a real-time video feed of the patient's larynx, other patient anatomy, or other objects or structures in the upper airway of the patient, as captured via a camera of the video laryngoscope and displayed on a display screen of the video laryngoscope. The video feed may assist a medical professional to visualize the patient's airway and facilitate manipulation and insertion of a tracheal tube.

Laryngoscopes may be fitted with different blades. Blades vary in shapes and sizes to accommodate for different patient anatomy. For example, a pediatric patient may use a smaller blade to fit a smaller airway, and an adult patient may use a larger blade to fit a larger airway. Blades with different curvature may provide ease of operability and/or different viewing angles of patient anatomy when placed inside of the airway.

Different blades may refract and/or reflect light differently. Thus, during intubation of a patient, use of common lighting and camera settings for different blades may not provide desirable or optimal viewing of patient anatomy. Additionally, different blade types may cause more or less obscuring of the camera of the video laryngoscope. Further, the use of different blades for treatment of a patient can lead to challenges with patient care and record-keeping. For example, later patient treatment may benefit from referencing which blade(s) were used to intubate a patient. Additionally, the use of different blades for a common video laryngoscope may cause challenged with tracking inventory of blades in a care facility.

Provided herein are systems and methods for blade detection with a video laryngoscope. The video laryngoscope may be capable of identifying a blade that has been coupled to the video laryngoscope. Identification of the blade may be based on an image captured by a camera of the video laryngoscope. In particular, a size and shape of obfuscation along a border of the image may be associated with different blades. For example, different blades have different sizes and curvatures that affect the size and shading of the blade-specific image obfuscation. The blade identification may be automatic. Blade identification may be performed via image recognition rules and/or machine learning (ML) models. Additionally, an image may be the only information used to determine blade identification. Based on the blade identification, various settings of the video laryngoscope may be adjusted (e.g., lighting or camera settings). The blade identification may be sent to a facility computer for maintaining patient records, procedure records, and/or inventory. Additionally, the blade identification may be used in determining a video classification of intubation (VCI) score.

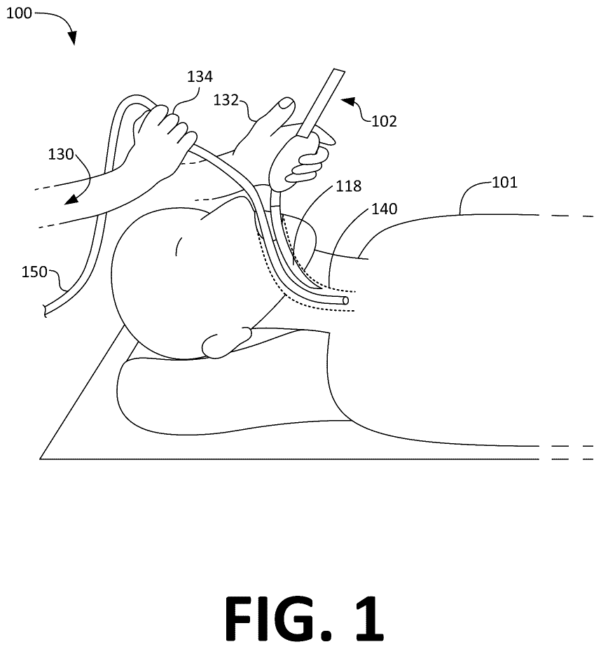

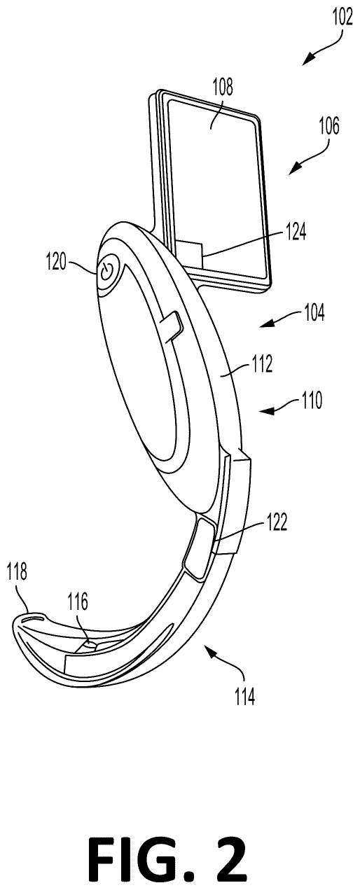

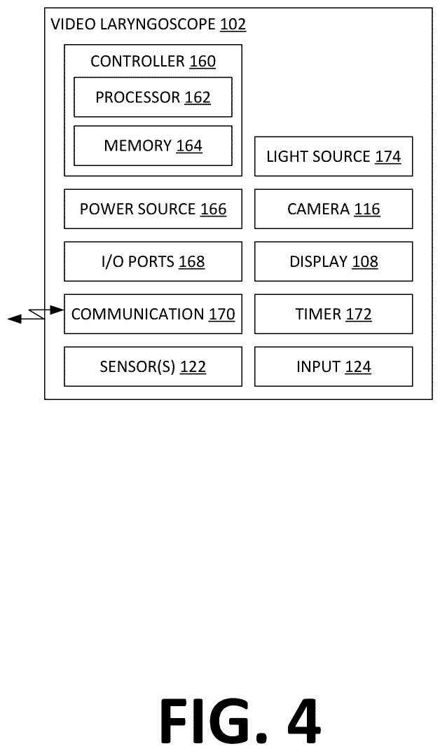

FIG. 1 shows an example patient environment 100 including a video laryngoscope 102. The patient environment 100 may be any room where an intubation is being performed, such as a medical suite in a hospital or other care setting, an operating or other procedure room, patient recovery room, an emergency intubation setting, or other environments. As described herein, the video laryngoscope 102 may be used for airway visualization of a patient 101 and/or blade identification, based on an image from a camera 116 of the video laryngoscope 102. The video laryngoscope 102 may be positioned in a patient's airway 140 concurrently with an endotracheal tube 150 (or tool to place the tube, such as an introducer). Aspects of the video laryngoscope 102 are further shown in FIGS. 2 and 4.

In FIG. 1, a medical professional 130 is shown holding a video laryngoscope 102 in a first hand 132 (e.g., a left hand 132 of the medical professional 130) and an endotracheal tube 150 in a second hand 134 (e.g., a right hand 134 of the medical professional 130). As further described herein, the video laryngoscope 102 may be positioned in the airway 140 of the patient 101 to manipulate and/or visualize the patient's airway 140, such as with an arm 114 or blade 118. Visualization of the airway 140 of the patient 101 may include viewing patient's anatomy (e.g., larynx, trachea, esophagus, vocal cords, etc.) with a camera 116 of the video laryngoscope 102. The medical professional 130 may move the tube 150 proximally (e.g., retract the tube 150) or distally (e.g., advance the tube 150), while watching the resulting images from the camera 116 of the video laryngoscope 102 on the display 108 of the video laryngoscope 102.

The images acquired from the camera 116 of the video laryngoscope 102 may include a portion of a blade 118 coupled to the video laryngoscope 102. For example, a portion of the blade 118 may partially obscure the camera 116. This partial obfuscation may be located along an upper border and/or upper corner of the images acquired from the camera 116. The appearance of the blade-associated obfuscation may be used to recognize and/or identify the type of blade 118 coupled to the video laryngoscope 102. For example, different sizes and curvatures of a blade 118 may result in different sizes, shapes, and shading of blade-associated obfuscation of the camera 116. The images used for blade identification may be raw or uncropped images. Alternatively, the images used for blade identification may be the cropped images displayed at the display 108 of the video laryngoscope.

Identification of a blade 118 by the video laryngoscope 102 may be based on a single image captured by the camera 116 of the video laryngoscope 102. The image may be a real-time, still-shot frame from a real-time video feed of a camera, such as a camera 116 of a video laryngoscope 102. Recognition or identification of the blade 118 from the single frame may be based on image recognition rules (e.g., coded heuristics or rule-based algorithms) or artificial intelligence (AI) algorithms and/or machine learning (ML) models (e.g., trained). The single frame may be the only input into image recognition rules or algorithms/model. If using a trained ML model, the model may be a neural network, such as a deep-learning neural network or convolutional neural network, among other types of AI or ML models. Other types of models, such as regression models, may also or alternatively be used. Training of the model may be based on one or more still-shot images associated with different blades. The trained model may receive and classify the single frame input into one of a finite quantity of blade identifications, trained based on comparisons or analysis of the sets of still-shot training images.

Blade identification can be performed on the video laryngoscope 102 itself and in real time (e.g., low latency). Because blade identification is based on image analysis, blades currently available on the market may be recognized without changing or updating the physical structure and/or components blades (e.g., RFID chip, barcode, laryngoscope-specific identification, etc.). Further, when new blades are created, the technology discussed herein may be updated with additional training to recognize the new blade without any changes to the hardware of either the video laryngoscope 102 or the blade. Additionally, no user input is required for blade identification. Blade identification may be performed automatically by the video laryngoscope 102.

The blade identification may be used in a variety of ways. The blade identification may be sent or transmitted to a remote device (e.g., care-facility computer) for record-keeping. This may include associating the blade identification with records of a procedure, recording which tools were used in a procedure, blade inventory, whether the patient 101 required or desired use of a specific blade type (e.g., a hyper-angulated blade, curved blade, or straight blade). Additionally or alternatively, various settings of the video laryngoscope may be changed or adjusted, based on the blade identification. Settings may include lighting settings (e.g., brightness of one or more light-emitting diodes (LEDs)) and/or camera settings. Camera settings may include gain, color, high dynamic range, crop region, backlight, contrast, or any other camera setting. Regarding adjustment of gain, in some situations a reflection of light or glare (such as by different blades 118 coupled to the video laryngoscope 102) may cause a bright spot in an image acquired by the camera. Accordingly, when a blade 118 is identified in an image, a gain map for the image can be changed to reduce any bright spot(s) caused by a reflection of that particular blade (e.g., a gain map may be associated with each possible blade identification).

Image analysis for blade identification may persist in a continuous loop. In a continuous loop analysis, contemporaneous image frames may be analyzed in real time. For example, each image frame of a video feed (e.g., frames acquired at 30 frame per second) may be analyzed. In another example, a subset of the total image frames of a video feed may be analyzed. For instance, every second, third, fourth, etc. frame may be analyzed. Alternatively, image frames may be analyzed in preset intervals (e.g., every 0.1 seconds, every 0.2 seconds, etc.) as may be tracked by a timer (e.g., timer 172 discussed in FIG. 4) of the video laryngoscope 102. As another alternative, image frames may be analyzed based on events, such as a power-on of the video laryngoscope 102, full camera obfuscation (e.g., which may be associated with removing or attaching of a blade 118), a user indication, or other indication that a blade has been moved or changed.

FIG. 2 shows a perspective view of the video laryngoscope 102. As shown, the video laryngoscope 102 has a body 104 (e.g., reusable body). The body 104 includes a display portion 106 having a display screen 108 that is configured to display images and/or other data, a handle portion 110 having a handle 112 that is configured to be gripped by the medical professional during the laryngoscopy procedure, and an elongate portion or arm 114 that supports a camera 116 and light source (e.g., light-emitting diodes (LEDs)) that is configured to obtain images, which may be still-shot images and/or moving images (e.g., a video feed). The camera 116 and light source may be incorporated on the distal end of the arm 114. The light source may be provided as part of the camera 116 or separate from the camera 116 on the blade 118 or arm 114.

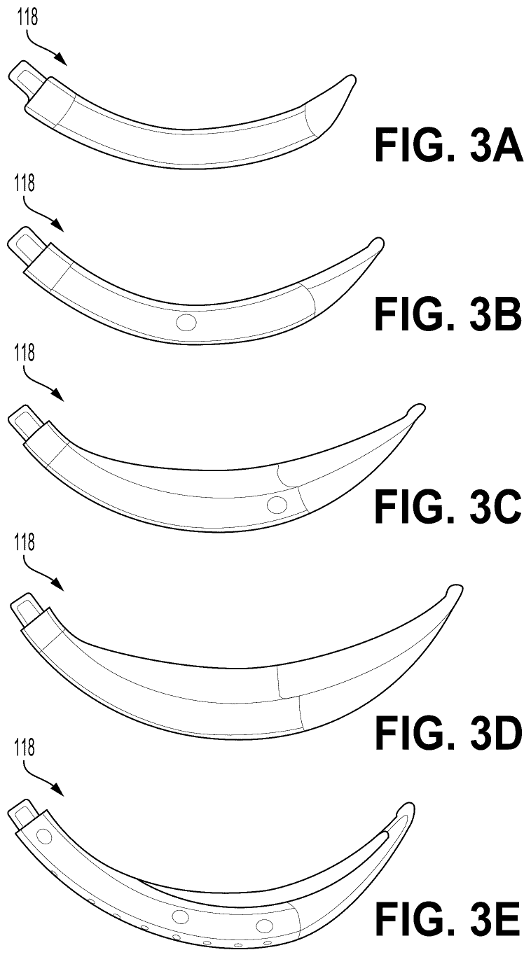

In examples, the display portion 106 and the handle portion 110 may not be distinct portions, such that the display screen 108 is integrated into the handle portion 110. In the illustrated embodiment, an activating cover, such as a removable laryngoscope blade 118 (e.g., activating blade, disposable cover, sleeve, or blade), is positioned about the arm 114 of the body 104 of the laryngoscope 102. Some examples of blades 118 are further shown in FIGS. 3A-3E. Together, the arm 114 of the body 104 and the blade 118 form an insertable assembly that is configured to be inserted into the patient's oral cavity. It should be appreciated that the display portion 106, the handle portion 110, and/or the arm 114 that form the body 104 of the laryngoscope 102 may be fixed to one another or integrally formed with one another (e.g., not intended to be separated by the medical professional during routine use) or may be removably coupled to one another (e.g., intended to be separated by the medical professional during routine use) to facilitate storage, use, inspection, maintenance, repair, cleaning, replacement, or interchangeable parts (e.g., use of different arms or extensions with one handle portion 110), for example.

The handle 112 and/or arm 114 may include one or more sensors 122 capable of monitoring functions (e.g., different, additional, and/or advanced monitoring functions). The sensors 122 may include a torque sensor, force sensor, strain gauge, accelerometer, gyroscope, magnet, magnetometer, proximity sensor, reed switch, Hall effect sensor, etc. disposed within or coupled to any suitable location of the body 104. The sensors 122 may detect interaction of the video laryngoscope 102 with other objects, such as a blade 118, physiological structures of the patient (e.g., teeth, tissue, muscle, etc.), or proximity of a tube, introducer, or other tool.

The laryngoscope 102 may also include a power button 120 that enables a medical professional to power the laryngoscope 102 off and on. The power button 120 may also be used as an input device to access settings of the video laryngoscope 102. Additionally, the video laryngoscope 102 may include an input button, such as a touch or proximity sensor 124 (e.g., capacitive sensor, proximity sensor, or the like) that is configured to detect a touch or object (e.g., a finger or stylus). The touch sensor 124 may enable the medical professional operating the video laryngoscope 102 to efficiently provide inputs or commands, such as inputs to indicate a change in a blade 118, inputs to transmit blade identification information, inputs that cause the camera 116 to obtain or store an image on a memory of the laryngoscope, and/or any other inputs relating to function of the video laryngoscope 102.

FIGS. 3A-3E show example blades 118 for the video laryngoscope 102. Although the example blades 118 shown in FIGS. 3A-3E are curved blades, blades of different shapes are appreciated (e.g., straight blades). FIGS. 3A-3D show different sizes of curved MacIntosh blades. In particular, FIG. 3A shows a MAC1 blade, which is a small blade that may be used for intubation of neonatal and/or infant patients. FIG. 3B shows a MAC2 blade, which is larger than the MAC1 blade. FIG. 3C shows a MAC3 blade, which is larger than the MAC2 blade, and FIG. 3D shows a MAC4 blade, which is larger than the MAC 3 blade. FIG. 3E shows a hyperangulated blade, sometimes referred to as an x-blade, which has more dramatic curvature than the other blades 118 shown in FIGS. 3A-3D.

The size and curvature of each blade 118 may cause different refraction of light from a light source of the video laryngoscope 102. For example, the MAC1, MAC2, MAC3, MAC4, and x-blade shown may each refract light differently and may result in different lighting and/or shading of patient anatomy when the blade is inserted into the patient airway. Additionally, the size and shape of the blade may cause more or less obfuscation of the camera 116 of the video laryngoscope 102. Based on blade identification, lighting settings and/or camera settings may be adjusted for more desirable viewing of patient anatomy. For example, the video laryngoscope may change a brightness of lighting (e.g., brighter or dimmer) to cause a desirable amount of light to be refracted and/or reflected into the patient anatomy, based on which blade is recognized. In another example, camera settings may be adjusted to reduce glare in a front portion of the patient anatomy while lighting the background. In a further example, the camera image may be cropped differently to remove a some or all of the blade-specific camera obfuscation from the image displayed at the display 108 of the video laryngoscope 102.

FIG. 4 is a block diagram of components of the video laryngoscope 102. As shown, the video laryngoscope 102 may include various components that enable the video laryngoscope 102 to carry out the techniques disclosed herein. For example, the video laryngoscope 102 may include the display screen 108, the camera 116, a light source 174 (e.g., which may integrated into the camera or separate from the camera), sensor(s) 122, and input (e.g., touch sensor) 124, as well as a controller 160 (e.g., electronic controller), one or more processors 162, a hardware memory 164, a power source (e.g., battery) 160, input/output (I/O) ports 168, a communication device 170, and a timer 172. In some embodiments, the timer 172 may track relative time (e.g., a start time, an end time, a frequency of image frame sampling), which may be referenced to acquire still-shot input images for analysis and/or determine an outcome of intubation (e.g., for determining a VCI score).

The communication device 170 may enable wired or wireless communication. The communication devices 170 of the video laryngoscope 102 may communicatively couple with communication devices of a remote device (e.g., a care-facility computer) to allow communication between the video laryngoscope 102 and the remote device (e.g., sending blade identification information). Wireless communication may include transceivers, adaptors, and/or wireless hubs that are configured to establish and/or facilitate wireless communication with one another. By way of example, the communication device 170 may be configured to communicate using the IEEE 802.15.4 standard, and may communicate, for example, using ZigBee, WirelessHART, or MiWi protocols. Additionally or alternatively, the communication device 170 may be configured to communicate using the Bluetooth standard or one or more of the IEEE 802.11 standards.

In some examples, the video laryngoscope 102 include electrical circuitry configured to process signals, such as signals generated by the camera 116 or light source 174, signals generated by the sensor(s) 122, and/or control signals provided via inputs 124 or automatically. The processors 162 may be used to execute software. For example, the processor 162 of the video laryngoscope 102 may be configured to receive signals from the camera 116 and light source 174 and execute software to acquire an image, analyze an image, identify a blade, etc.

The processor 162 may include multiple microprocessors, one or more “general-purpose” microprocessors, one or more special-purpose microprocessors, and/or one or more application specific integrated circuits (ASICS), or some combination thereof. For example, the processor 162 may include one or more reduced instruction set (RISC) processors.

The hardware memory 164 may include a volatile memory, such as random access memory (RAM), and/or a nonvolatile memory, such as read-only memory (ROM). It should be appreciated that the hardware memory 164 may include flash memory, a hard drive, or any other suitable optical, magnetic, or solid-state storage medium, other hardware memory, or a combination thereof. The memory 164 may store a variety of information and may be used for various purposes. For example, the memory 164 may store processor-executable instructions (e.g., firmware or software) for the processor 162 to execute, such as instructions for processing signals generated by the camera 116 to generate the image, provide the image on the display screen 108, analyze an image via a trained model, identify a particular blade type and/or blade size from the image, adjusting camera and/or lighting settings, etc. The hardware memory 164 may store data (e.g., acquired images, training images, blade identifications, image recognition rules, AI or ML algorithms, trained models, tags or labels, mode data, etc.), instructions (e.g., software or firmware for generating images, storing the images, analyzing the images, identifying the blade types, adjusting settings, etc.), and any other suitable data.

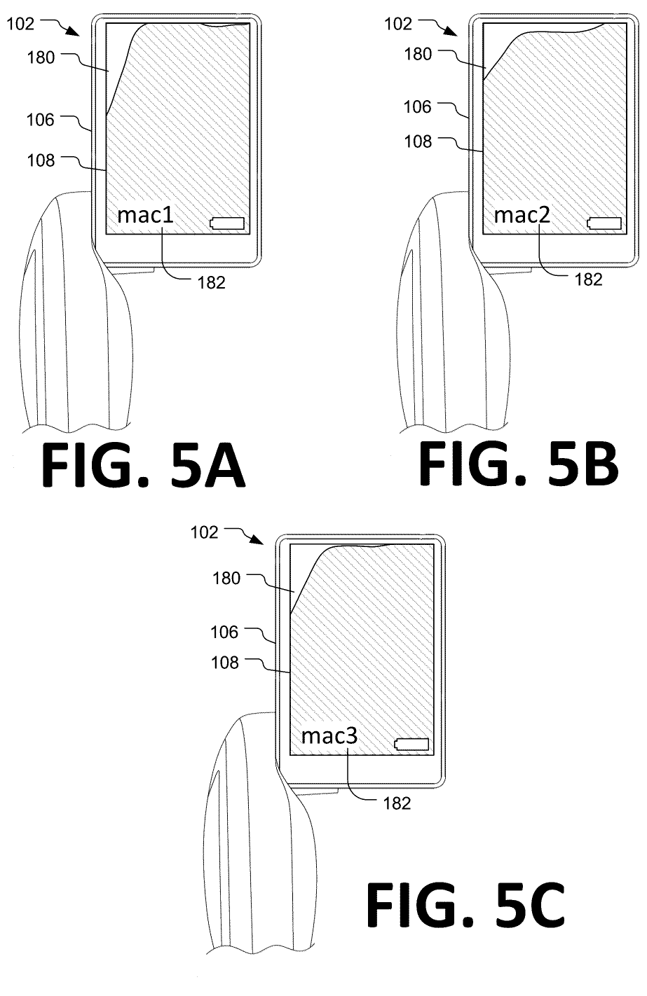

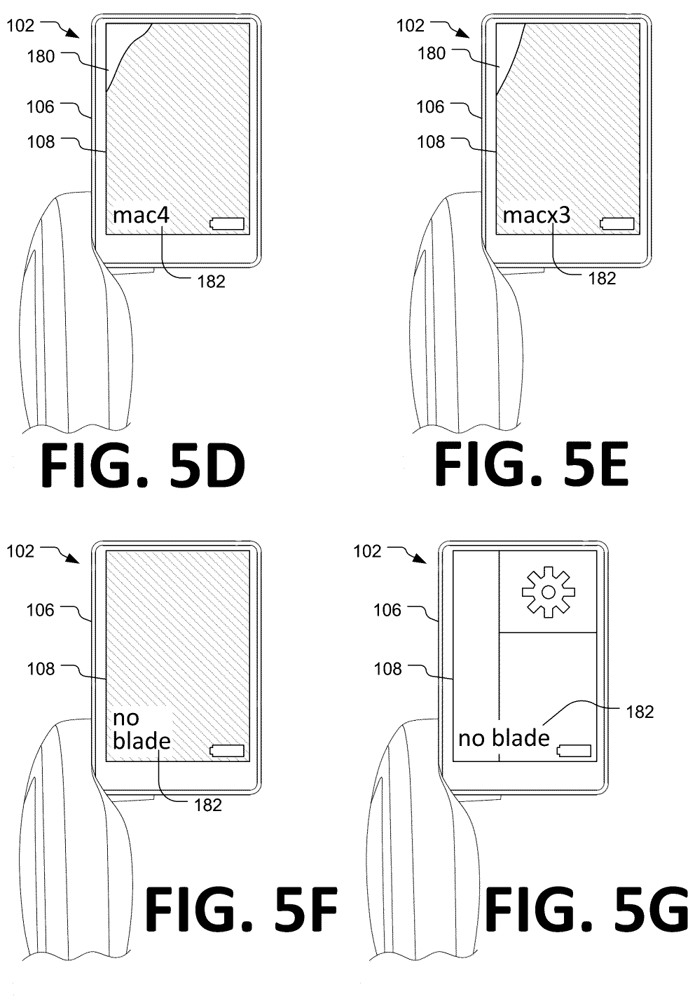

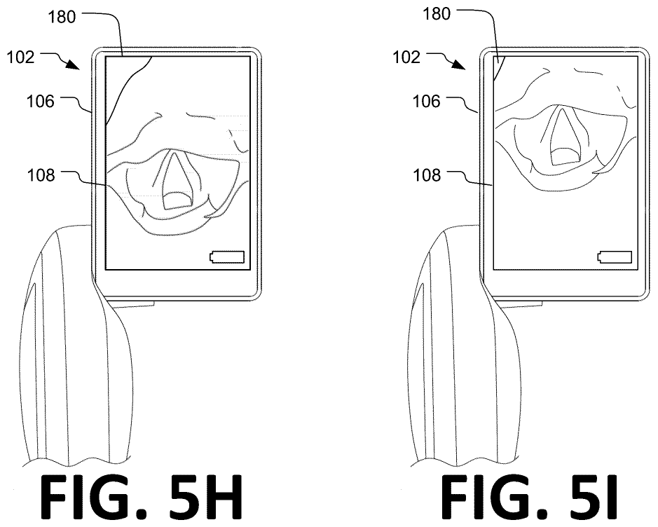

FIGS. 5A-5I show example display screens 106 of a video laryngoscope 102 with blade size detection. As described herein, a camera 116 of the video laryngoscope 102 captures an image including a portion of a blade. The captured images are displayed on a display 108 of the video laryngoscope 102. In some examples, the raw images captured by the camera 116 may be larger than the images displayed at the display 108 of the video laryngoscope 102. In such examples, the captured images may be cropped to a crop region for display. Some or all of the image frames (cropped or uncropped) may be analyzed to recognize or identify a blade 118 coupled to the video laryngoscope 102.

A visual indicator 182 may be overlaid on the display 108. The visual indicator 182 may provide information relating to blade identification, detection, and/or placement. For example, the visual indicator 182 may include the identification of the blade (e.g., size, curvature, brand, style, unidentified, etc.), if a blade is not detected (e.g., no blade), and/or warnings and/or cautions regarding blade placement (e.g., camera obscured by blade, blade loose/not secured, etc.). The visual indicator 182 may include information, instructions, or warnings.

For example, in an instance where no blade is detected, a warning may provide “no blade” or “blade missing,” etc. In another example, when no blade is detected, non-clinical functionality of the video laryngoscope 102 may be enabled. Non-clinical functionality may include a display of various visual information at the display 108. For instance, non-clinical visual information may include a settings menu, device settings, on-device video replay, etc. Display of non-clinical visual information may exclude images captured currently, in real-time from the camera 116 of the video laryngoscope 102 (e.g., a clinical image). Alternatively, when no blade is detected, no image or other functionality may be displayed, other than a visual indication of no blade. When a blade is detected, the video laryngoscope 102 may automatically display a clinical image (e.g., a video feed currently captured by the camera 116 of the video laryngoscope 102) at the display 108. Operation of the video laryngoscope without a blade may not properly open a patient's airway, and may not allow for visualization of the airway with the camera (e.g., the camera may be obstructed by patient anatomy), in addition to other operational constraints.

As another example, in an instance where a loose blade is detected, a warning may provide “loose blade,” “blade not secure,” “check blade attachment,” etc. Operation of the video laryngoscope with a loose/unsecured blade may result in the blade decoupling from the video laryngoscope before, during, or after a procedure. Decoupling may cause total camera obfuscation, loss of the blade prior to insertion into the airway (e.g., no blade detected), and/or decoupling of the blade when removing the video laryngoscope from the patient's airway (e.g., the blade may be accidentally left inside the patient after intubation). The visual indicator 182 may be displayed concurrently with an image of patient anatomy being captured by the camera of the video laryngoscope 102.

Although the visual indicator 182 is shown in the form of text in FIGS. 5A-5G, any visual indicator 182 is appreciated, including images, symbols, text, abbreviations, blurring, changes in brightness and/or contrast, etc. In some examples, such as those shown in FIGS. 5H-5I, no visual indicator 182 may be displayed relating to blade identification.

FIGS. 5A-5E show different blade-associated camera obfuscations 110, which may result from different blades 118 being coupled to the video laryngoscope 102. For example, the different displays 108 shown in FIGS. 5A-5E may correspond with the different blades 118 shown in FIGS. 3A-3E. As shown, the different blades may each result in a unique blade-associated camera obfuscation 180. Each blade-associated camera obfuscation 180 may have a unique size, shape, and/or shading pattern (not shown).

FIG. 5F shows an example display screen 108 showing an image with no blade detected. This may be determined as a part of blade identification (e.g., no blade may be an output of image recognition rules and/or an output of an ML model). Alternatively, blade detection may be part of a preliminary analysis, prior to blade identification determination. In an environment where no blade is detected, the visual indicator 182 may indicate “no blade” and/or other alerts or warnings. For example, the visual indicator 182 may provide instructions to check for a blade prior to intubating.

FIG. 5G shows another example display screen 108 showing an image with no blade detected. Unlike FIG. 5F, which shows the visual indicator 182 overlaid onto a clinical image shown on the display 108, FIG. 5G shows a display of non-clinical functionality. As an example, FIG. 5G shows a settings menu at the display 108 when no blade is detected. The visual indicator 182, indicating “no blade” may be overlaid on the non-clinical functionality. When a blade is detected, a clinical image may be automatically displayed at the display 108 of the video laryngoscope 102 (such as the displays 108 shown in FIGS. 5A-5E and 5H-5I).

FIGS. 5H-5I show example display screens 108 including different cropped regions for an image acquired by the camera 106 of the video laryngoscope 102. In examples, a raw or full-size image captured by the camera 106 may be cropped based on a blade identification. For example, in FIG. 5H, the blade-associated camera obfuscation 180 obscures a large portion of the image provided on the display 108 (e.g., a blade-associated camera obfuscation 180 associated with a MAC4 blade). When the video laryngoscope 102 identifies the blade (e.g., identification of a MAC4 blade, based on the image), the video laryngoscope 102 may adjust one or more settings to improve the view of the image at the display 108. In the example provided in FIG. 5I, the crop region of the image is adjusted to omit a portion of the blade-associated camera obfuscation 180. Additionally, in FIG. 5I, the brightness of the lighting is adjusted to improve illumination of patient anatomy.

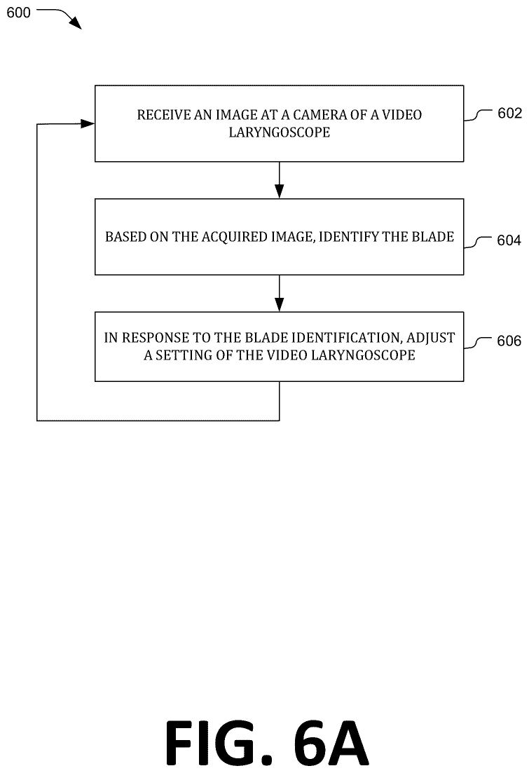

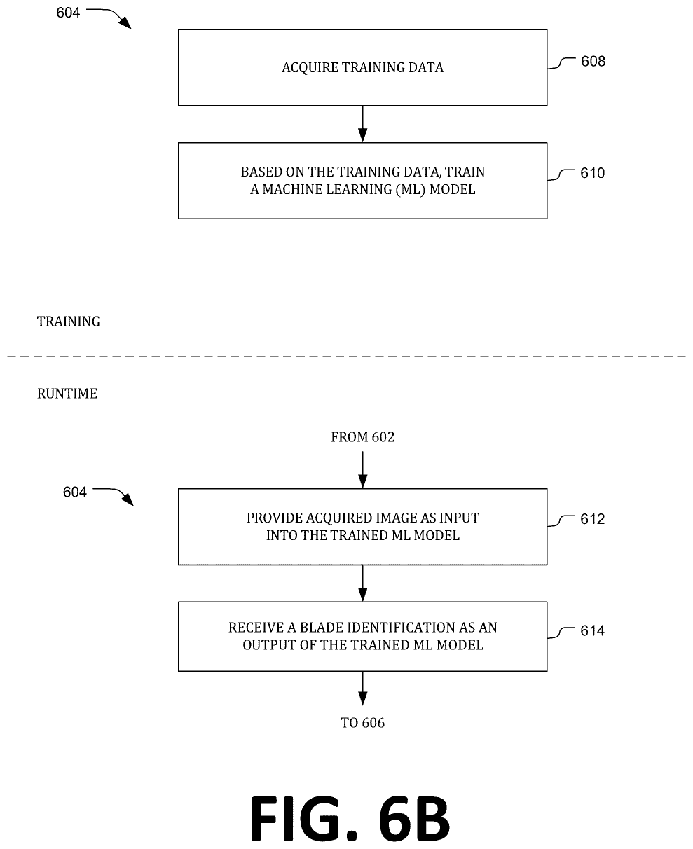

FIGS. 6A, 6B, 7, and 8 show example methods according to the disclosed technology. The example methods include operations that may be implemented or performed by the systems and devices disclosed herein. For example, the video laryngoscope 102, depicted in at least FIGS. 1, 2, and 4, may perform the operations described in the methods. In addition, instructions for performing the operations of the methods disclosed herein may be stored in a memory of the video laryngoscope (e.g., system memory 164 described in FIG. 4).

FIG. 6A shows an example method 600 for blade detection with a video laryngoscope. At operation 602, an image is received at a camera of a video laryngoscope. The image may be a raw/full-sized image from the camera, or may be a cropped version of the image fitted for a display of the video laryngoscope. The image may include a portion of a blade coupled to the video laryngoscope in the form of a blade-associated camera obfuscation. The image may be prior to the blade's insertion into a patient's airway (e.g., no patient airway anatomy in the image). Alternatively, the image may be acquired during insertion, before intubation, during intubation, or after intubation, such that patient anatomy is also included in the image.

At operation 604, the blade is identified. The blade may be identified based on the acquired image. Blade identification may be automatic and/or in real time. As otherwise described herein, a blade may be identified or recognized based on a blade-associated camera obfuscation in the image. For example, the blade may be identified based on size, shape, and/or shading of the blade-associated camera obfuscation.

One example of operation 604 is further described with respect to FIG. 6B. As shown, the blade identification may be determined by image recognition rules, AI algorithms, and/or ML models. The machine learning (ML) model may be trained (e.g., as described in operations 608, 610) prior to runtime operations (e.g., operations 612, 614).

During training of the ML model, operations 608, 610 may be performed. Training of the trained model may occur prior to the trained model's deployment/installation on the video laryngoscope. At operation 608, training data is acquired. The training data may include a large set or sets of images that are labeled with respective corresponding classifications (e.g., the blade type connected to the laryngoscope when the images were captured). The training data may be labeled with the corresponding classes via manual classifications or through other methods of labeling images. Classifications may include blade identifications. For example, a first set of training images may be labeled with a first blade identification, a second set of training images may be labeled with a second blade identification, a third set of training images may be labeled with a third blade identification, etc. The labels may include blade identifications such as MAC 1, MAC 2, MAC 3, MAC 4, MAC X1, MAC X2, MAC X3, MAC X4, size 1 straight blade, size 2 straight blade, size 3 straight blade, size 4 straight blade, etc.

In some examples, the training data images may be a portion of raw/full-sized images acquired by a camera of a video laryngoscope. For example, training images may be cropped images from a camera of a video laryngoscope, with the crop region including a portion of the acquired images in which a blade-associated camera obfuscation would be visible. For instance, a crop region for the training data images may be an upper half, upper third, upper fourth, or upper portion of the acquired image, an upper left quadrant, etc. Limiting the training data to relevant blade detection regions may remove noise from the training data.

At operation 610, the ML model is trained, based on the training data. Training the ML model with the training data set may include use of a supervised or semi-supervised training method or algorithm that utilizes the classified images in the training data. Once the trained model is generated, the trained model may be used to determine blade identification in real time.

After the ML model is trained, the trained ML model may be used in performing operations 612, 614 during runtime. At operation 612, images acquired by a camera of a video laryngoscope (e.g., the images received in operation 602 in FIG. 6A) are provided into the trained ML model. The acquired images may be provided as an input into the trained ML model in real time. If the training data includes cropped images, then the live acquired images used during runtime may be cropped accordingly prior to being provided as an input into the trained ML model.

At operation 614, a blade identification is received as an output of the trained ML model. The input image (e.g., the acquired image, which may be cropped appropriately), may be received by the trained ML model and classified into one of the trained classes. In some examples, when classifying an input image, the trained ML model may also output confidence score associated with the classification. If a confidence score determined for an input image classification is below a confidence threshold, of the blade may be considered or determined to be an unidentified blade (e.g., an indication that the detected blade does not fit into one of the trained classifications). In addition, in some examples, the ML model is also trained on a set of captured, labeled images where no blade is attached to the video laryngoscope. In such examples, where an image is captured at runtime by a laryngoscope with no blade, the image may be classified as no blade or blade missing. Further, in some examples, the ML model is also trained on a set of captured, labeled images where a loose blade is attached to the video laryngoscope (e.g., the blade is not securely attached to the video laryngoscope). In such examples, where an image is captured at runtime by a laryngoscope with a loose blade, the image may be classified as loose blade or unsecured blade. The outputted blade identification (or identification of no blade) from the trained ML model may then be used by the video laryngoscope (e.g., as described at operation 606 in FIG. 6A).

Returning to FIG. 6A, at operation 606, a setting of the video laryngoscope is adjusted. The setting may be adjusted based on, or in response to, the blade identification at operation 604. Settings of the video laryngoscope that may be adjusted include brightness of lighting (e.g., brightness of one or more LEDs), camera settings (e.g., gain, color, backlight brightness, crop region, contrast, etc.), or other settings that may enhance a quality of the image received at the camera of the video laryngoscope. Desired settings may be pre-determined for each possible blade identification. For example, after blade identification, the video laryngoscope may perform a lookup to determine appropriate settings. For instance, a first blade identification may be associated with a first set of settings (e.g., a first brightness of lighting and a first crop region of the camera image) and a second blade identification may be associated with a second set of settings (e.g., a second brightness of lighting and a second crop region of the camera image). Alternatively, the output of the image recognition rules, AI algorithms, and/or ML models may include both the blade identification and instructions for the video laryngoscope to adjust settings.

Operations 602-606 may repeat as required or desired. For example, different image frames may be analyzed over time (e.g., after a set quantity of frames, after a certain time, when the video laryngoscope is powered on, after a full camera obfuscation is detected, as directed by an indication at a user interface of the video laryngoscope, etc.). When a new blade is identified at operation 604, the settings of the video laryngoscope may be adjusted accordingly. For instance, a new image may be acquired by the camera of the video laryngoscope at operation 602. At operation 604, a new blade identification may be determined, such as by providing the new image as an input to a trained machine learning model and receiving, as output from the trained machine learning model, a new blade identification for the new image. At operation 606, based on the new blade identification, at least one setting of the video laryngoscope (e.g., lighting setting and/or camera setting) may be re-adjusted (e.g., if the new blade identification is different from the previous blade identification).

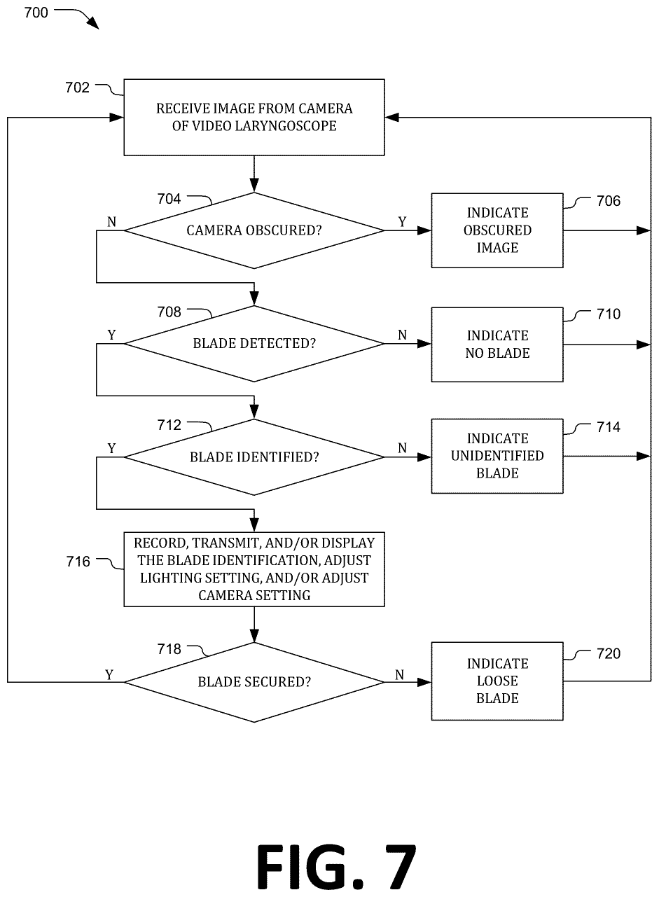

FIG. 7 shows another example method 700 for blade detection with a video laryngoscope. At operation 702, an image is received from a camera of a video laryngoscope. Operation 702 may be similar to operation 602 described in FIG. 6. The image may include a portion of a blade coupled to the video laryngoscope in the form of a blade-associated camera obfuscation.

At determination 704, camera obscuring and/or interference is determined. In this determination 704, a total obfuscation of the camera, beyond the expected blade-associated camera obfuscation, is evaluated. Total camera obscuring may result from a blade that is coupled to the video laryngoscope but not properly secured to the video laryngoscope (e.g., the blade is loose and blocking the camera). Total obscuring may be determined based on a glare or excessive brightness across all or a majority of the image captured at operation 702. If, at determination 704, the camera is determined to be obfuscated, then the method flows to operation 706 where an indication of an obfuscated image is provided. The indicator may be a visual indicator overlaid on the image at a display of the video laryngoscope. In some examples, the indicator may prompt an operator to check placement of the blade. In some examples, no indication (e.g., no visual indicator) is provided because an obscured screen may appropriately alert the operator of the video laryngoscope. Operations 702-706 may repeat as required or desired. For example, a new image may be received until the camera is no longer obscured.

If, alternatively, at determination 704 the camera is determined to not be totally obscured, the method 700 flows to determination 708. At determination 708, blade detection is determined. As described above, a blade-associated camera obfuscation is predicted or probable in certain parts of the acquired image (e.g., around a border, along an upper edge, in an upper corner, etc.) and has a predictable shape. If no blade-associated camera obfuscation is detectable, then a determination may be made that no blade is coupled to the video laryngoscope. If, at determination 708, no blade is detected, the method 700 flows to operation 710 where an indication of no blade is provided. Additionally or alternatively, the indicator may prompt or alert an operator to check for a blade prior to intubation. When no blade is detected, a clinical image (such as that shown in FIG. 5F) or a non-clinical functionality (such as that shown in FIG. 5G) may be displayed at a display of the video laryngoscope. With display a clinical image or non-clinical functionality, the indication of no blade may be overlaid on the provided display. A non-clinical image may include a settings menu, device settings, on-device video replay, etc. Alternatively, when no blade is detected, the indication of no blade may be the only visual information displayed (e.g., no clinical image displayed and no non-clinical functionality displayed, such that nothing is displayed other than the indication of no blade). Operations 702-710 may repeat as required or desired. For example, a new image may be received until a blade is detected.

If, alternatively, at determination 708 a blade is detected, the method 700 flows to determination 712. At determination 712, it is determined if the blade (e.g., the blade detected at determination 708) is identified. In some examples, blade identification may include a determination of blade detection described in determination 708 (e.g., determinations 708 and 712 may be part of a same determination). As otherwise described herein, a blade may be identified or recognized based on a blade-associated camera obfuscation in the image. For example, the blade may be identified based on size, shape, and/or shading of the blade-associated camera obfuscation. The blade identification may be determined by image recognition rules, AI algorithms, and/or ML models. If the blade-associated camera obfuscation does not fit an image recognition rule and/or is not recognized by AI algorithms/ML models, then the blade is determined to be not identifiable. For example, the rules, algorithms, and/or models may not be structured or trained based on every available blade couplable to the video laryngoscope, such as new blades becoming available on the market, incompatible blades, third-party blades, etc. If, at determination 712, the blade is not identified, the method 700 flows to operation 714 where an indication of an unidentified blade is provided. The indication may prompt the operator to manually provide the blade in client records and/or for inventory. Additionally or alternatively, the indicator may include a warning that the blade may not be compatible with some functionalities of the video laryngoscope (e.g., record-keeping, settings adjustment based on blade identification, etc.). Operations 702-714 may repeat as required or desired. For example, a new image may be received until a blade is identified.

If, alternatively, at determination 712 the blade is identified, the method 700 flows to operation 716. At operation 716 the blade identification may be recorded (e.g., saved with a file on the video laryngoscope), transmitted (e.g., to a remote device, such as for record-keeping and/or inventory), and/or displayed. Display of the blade identification may provide information for the operator and/or training for referencing if an appropriately sized blade has been selected for the patient. Additionally or alternatively, at operation 716, a setting of the video laryngoscope may be adjusted. For example, as described herein, a lighting setting and/or a camera setting may be adjusted.

At determination 718, secure attachment of the blade is determined. In some environments, a blade may be positioned such that the camera is not totally obscured (e.g., as described at determination 704), but not properly secured to the video laryngoscope. In such an example, the blade-associated camera obfuscation may be improperly located, slightly larger than expected, and/or cause different glare or shading in the acquired image. An unsecured blade may be accidentally left behind inside the patient's airway after the video laryngoscope is removed (e.g., after intubation). If, at determination 718, the blade is determined to be improperly positioned/coupled, the method 700 flows to operation 720 where an indication of improper/unsecure blade attachment is provided (e.g., loose blade). Additionally or alternatively, the indication may prompt the operator to check blade attachment, proceed with caution, and/or check patient after procedure. Operations 702-720 may repeat as required or desired. For example, a new image may be received until a blade is determined to be securely attached or properly positioned.

If, alternatively, at determination 718 the blade is determined to be securely attached or properly positioned, the method 700 may repeat operations 702-718. As described herein, different image frames may be analyzed over time. As a new blade is detected and/or identified, the blade identification may be recorded, transmitted, and/or displayed. Additionally or alternatively, a setting of the video laryngoscope may be adjusted. In an example where multiple blade identifications are determined for a use session of the video laryngoscope (e.g., during a same power-on session, intubation of the same patient, a single intubation session, closeness in time, etc.), each of the determined blade identifications may be recorded, transmitted, and/or displayed.

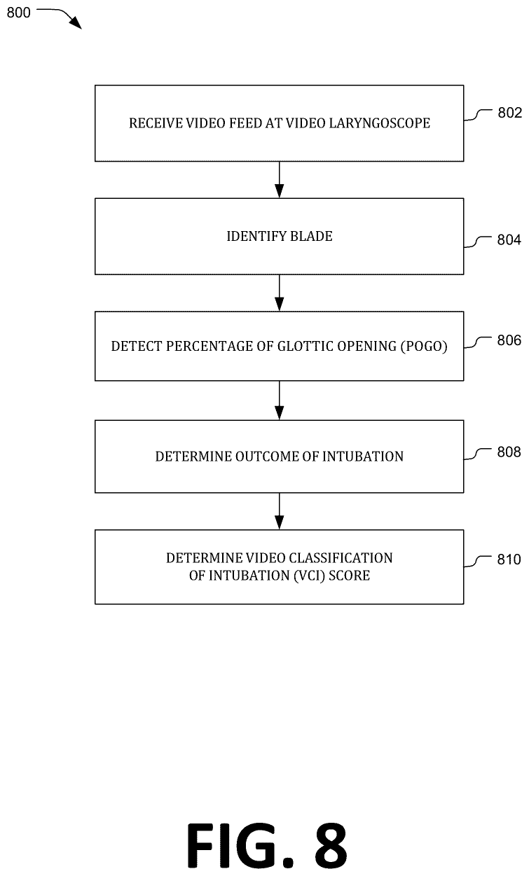

FIG. 8 shows an example method 800 for determining a video classification of intubation (VCI) score with a video laryngoscope. The video laryngoscope may automatically determine enough information about the intubation procedure to suggest a VCI score. The VCI score (vciscore.com/) is a way to quickly classify an airway and intubation with a letter, number, and letter. The first letter is the blade used (M=Macintosh, H-hyperangulated, S=straight), the number is the percentage of glottic opening (POGO) visible, and the last letter is whether the intubation succeeded (E=easy, D=difficult) or failed (F). For example, a VCI score of M75E is Macintosh blade, 75% open, Easy.

At operation 802, a video feed is received at a video laryngoscope. The video feed may be acquired by a camera of the video laryngoscope. The video feed may include images individually analyzable for determining components of the VCI score.

At operation 804, a blade is identified. The blade may be identified based on a first image from the video feed. The first image includes a portion of a blade coupled to the video laryngoscope in the form of a blade-associated camera obfuscation. Blade identification, based on the image, is further described herein. Blade identification may include one of the categories for determining a VCI score (e.g., M/H/S). In an example, the blade may be identified as a MAC3, associated with an “M.” In another example, the blade may be identified as a MACX3, associated with an “H.” As described herein, blade identification may be determined based on image recognition rules, AI algorithms, and/or ML models.

At operation 806, a percentage of glottic opening (POGO) is detected. The POGO may be identified based on a second image from the video feed. The second image includes the vocal cords of the patient (e.g., the second image is after the blade and the video laryngoscope are inserted into the airway of the patient). In some instances, the first image (as described in operation 804) and second image may be the same. The video laryngoscope may be capable of identifying patient anatomy (e.g., the vocal cords) and taking measurements of the patient anatomy to determine the POGO. The POGO may be one of a set percentage, such as 0%, 25%, 50%, 75%, and 100%. A POGO score of 100% is associated with visualization of the entire glottis from the anterior commissure of the vocal cords to the interarytenoid notch. If no portion of the vocal cords are visible, then the POGO score is 0%. In examples, the POGO may be determined by image recognition rules, AI algorithms, and/or ML models. For example, the second image may be provided as an input into a trained ML model. The trained ML model may be trained with training data, including large sets of images labelled with a classification. Classifications may include 0%, 25%, 50%, 75%, and 100%. Thus, the output of the trained ML model for the second image may be one of the trained classifications.

At operation 808, an outcome of intubation is determined. The outcome of intubation may be based on at least a third image, detecting proper placement of an endotracheal tube in the patient's airway. A score of easy (E) may result from an intubation per a manufacturer's recommendations. A score of failed (F) is a failure to properly place/pass the endotracheal tube. Anything in between easy and failed is a score of difficult (D). Difficulty is not determined based on the overall intubation process, but instead is based on the use of extra equipment (e.g., bougies, forceps, flexible scopes, etc.). Thus, if the video laryngoscope identifies any tools in the at least third image other than the blade and the endotracheal tube, then the intubation outcome score is difficult. Outcome of the intubation may be determined based on image recognition rules, AI algorithms, and/or ML models.

At operation 810, a video classification of intubation (VCI) score is determined. Based on the determinations made for blade identification, POGO, and intubation outcome in operations 804-808, a VCI score may be determined. The VCI score may be recorded, transmitted, and/or displayed, such as for record-keeping and/or training.

The techniques introduced above may be implemented for a variety of medical devices or devices where direct and indirect views are possible. A person of skill in the art will understand that the technology described in the context of a video laryngoscope for human patients could be adapted for use with other systems such as laryngoscopes for non-human patients or medical video imaging systems.

Those skilled in the art will recognize that the methods and systems of the present disclosure may be implemented in many manners and as such are not to be limited by the foregoing aspects and examples. In other words, functional elements being performed by a single component or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for blade size detection by a video laryngoscope, the method comprising:
acquiring an image, by a camera of the video laryngoscope, the image including a portion of a blade coupled to the video laryngoscope;
based on the acquired image, automatically identifying the blade; and
in response to the identification of the blade, adjusting at least one setting of the video laryngoscope.

2. The method of claim 1, wherein the at least one setting of the video laryngoscope is a camera setting, and wherein the camera setting is one of:
gain;
color;
backlight brightness; or
crop region.

3. The method of claim 1, wherein the at least one setting of the video laryngoscope is a brightness of lighting provided by the video laryngoscope.

4. The method of claim 1, wherein the blade identification includes at least one of a size of the blade or a curvature of the blade.

5. The method of claim 1, wherein automatically identifying the blade includes providing the image as an input to a trained machine-learning (ML) model.

6. The method of claim 1, wherein the acquired image is an uncropped image of the camera, and wherein a cropped version of the acquired image is displayed on a display of the video laryngoscope.

7. A video laryngoscope comprising:
a handle portion;
a display screen coupled to the handle portion;
a blade portion, coupled to the handle portion and coupled to a blade, configured to be inserted into a mouth of a patient;
a camera, positioned at a distal end of the blade portion, that acquires a video feed while the video laryngoscope is powered on;
a memory storing a trained machine-learning (ML) model; and
a processor that operates to:
receive an image of the video feed from the camera, the image including a portion of the blade;
provide the image as input to the trained ML model;
receive an output from the trained ML model in response to the input image, wherein the output includes an identification of the blade; and
transmit the blade identification to a remote device.

8. The system of claim 7, wherein the remote device is a hospital computer for maintaining at least one of inventory or patient records.

9. The system of claim 7, wherein the video laryngoscope further includes lighting on the blade portion, and wherein the processor further operates to adjust at least one of a lighting setting of the lighting or a camera setting of the camera, based on the blade identification.

10. The system of claim 7, wherein the blade identification is displayed on the display screen.

11. The system of claim 7, wherein the blade identification is determined automatically.

12. The system of claim 7, wherein the determination of the blade identification is based only on the image.

13. A method for blade size detection by a video laryngoscope, the method comprising:
acquiring one or more images using a camera of the video laryngoscope;
based on the one or more images:
identifying a blade coupled to the video laryngoscope;
determining a percentage of glottic opening (POGO) visible; and
determining an outcome of intubation; and
based on the blade identification, the POGO visible, and the outcome of intubation, determining a video classification of intubation (VCI) score.

14. The method of claim 13, wherein the blade identification is based on a first image of the one or more images that includes a portion of a blade coupled to the video laryngoscope; wherein the POGO visible is based on a second image of the one or more images that includes the portion of the blade and patient anatomy; and wherein the outcome of intubation is based on a third image.

15. The method of claim 13, wherein the one or more acquired images are provided as inputs into a trained machine learning (ML) model, and wherein at least one of the blade identification or the POGO visible are received as outputs of the trained ML model.
